(19) United States
(12) Patent Application Publication
(10) Pub. No.: US 2012/0000472 A1
(43) Pub. Date: Jan. 5, 2012

(54) ATHLETIC MOUTH GUARD AND BREATHING TRAINER

(76) Inventor: Michael J. Martucci, Ukiah, CA (US)

(21) Appl. No.: 13/174,618

(22) Filed: Jun. 30, 2011

Related U.S. Application Data
(60) Provisional application No. 61/360,373, filed on Jun. 30, 2010.

(51) Int. Cl. A61C 5/14; A61M 16/20

(52) U.S. Cl. 128/861; 128/205.24

(57) ABSTRACT

An athletic mouth guard and breathing trainer having arcuate buccal wall, an arcuate lingual wall spaced apart from the arcuate buccal wall, a tooth bed disposed between the arcuate lingual wall and arcuate buccal wall, a valve box including an air passage with an opening in the front portion of the arcuate buccal wall, and a one-way valve disposed in the valve box opening. The valve has an open position and a closed position and can be opened with an exhalation through the valve insert opening, though not with an inhalation, such that after exhalation the valve returns to a closed position, thus encouraging the user to inhale through the nose and to engage in circular breathing.

### CROSS REFERENCES TO RELATED APPLICATIONS

The present application claims the benefit of U.S. Provisional Patent Application Ser. No. 61/360,373, filed Jun. 30, 2010 (Jun. 30, 2010.)

### STATEMENT REGARDING FEDERALLY SPONSORED RESEARCH OR DEVELOPMENT

Not applicable.

### NAMES OR PARTIES TO A JOINT RESEARCH AGREEMENT

Not applicable.

### INCORPORATION-BY-REFERENCE OF MATERIAL SUBMITTED ON A COMPACT DISC

Not applicable.

### SEQUENCE LISTING

Not applicable.

### BACKGROUND OF THE INVENTION

1. Field of the Invention

The present invention relates generally to mouth guards, and more particularly to athletic mouth guards with breathing channels, and still more particularly to an athletic mouth guard with breathing training elements.

2. Background Discussion

It has long been known by boxers and martial arts practitioners that improper breathing during competition causes tension, fatigue, and possibly confusion. Accordingly, in practice competitors cultivate breathing patterns in which they inhale and exhale through the nose in steady breathing rhythms as long as possible, and when forced to do so by exertion, switch to inhaling through the nose and exhaling through the mouth through pursed lips. Steady deep breathing helps the fighter to remain relaxed and composed and is optimal for oxygenating muscles and the brain. Movements are easier, attention remains focused, judgment stays clear and reliable. Mouth breathing is contraindicated for two reasons: first, it tends to be quick and shallow; second, it may entail opening the mouth, even slightly, and this tends to allow the jaw to go slack and renders it vulnerable to a knockout punch and possible fracture. Clamping down on the mandatory mouthpiece and breathing through the nose eliminates that problem.

However, when engaged in burst of extreme anaerobic exertions or when functioning near maximal aerobic output for prolonged periods, it can be challenging to sustain steady rhythmical breathing. Thus, fighters have learned that there are also advantages to exhaling rapidly or in bursts when throwing punches or kicks, as this both complements the muscular exertion in the arms or legs and also naturally tenses the abdomen to absorb a counter punch or kick. It is therefore not uncommon to hear a hissing type sound when a fighter throws punches or kicks. It is not difficult to appreciate that such noises may be physiologically advantageous, but they also telegraph a fighter's moves. And if performed with a conventional mouth guard in place, such patterns entail relaxation in the jaw to produce sufficient clearance for the passage of air.

It is therefore desirable that fighters learn proper “circular” breathing patterns—in through the nose and out through the mouth—but to do so without introducing noise and to do so without relaxing the bite on the mouth guard.

To that end, at least two major manufacturers of boxing and mixed martial arts equipment have devised mouth guards with air flow channels. The products include the TAPOUT® mouth guards made by Tapout LLC, and the BRAIN PAD® mouth guards made by Everlast Sports Manufacturing Company of New York, N.Y. Each product includes conventional mouth guard features but introduces a center portion having air flow holes or channels. There is no apparatus includes in the air flow holes to affect either inhalation or exhalation. [TAPOUT® is a registered trademark of AGB Tapout LLC, of Toronto, Ontario. BRAIN PAD® is a registered trademark of WIPSS Products, Inc, of Conshohocken, Pa.]

While these two products are of very high quality, they suffer a singular disadvantage that appears not to have been appreciated by its developers: They each allow for unobstructed inhalation through the mouth, and therefore they do not encourage proper breathing rhythm. Each apparatus leaves unanswered the question of how to use the mouth guard to facilitate and train proper circular breathing.

A few breathing training devices have been developed quite recently. Among them, U.S. Pat. No. 6,722,360 to Doshi, which discloses a mouthpiece with a valve comprising a hinged flap to increase resistance to expiration, preferably with a lesser increase in flow resistance to inspiration.

U.S. Pat. Appl. Ser. No. 20030121520 to Parker et al. teaches a mouthpiece for use in association with nose continuous positive airflow pressure (CPAP) systems to prevent leakage from the mouth during CPAP treatment, and thereby to treat sleep apnea. The mouthpiece is a thin flexible disk that fits between the user's lips and teeth and seals the mouth around the interior of the lips to prevent air from escaping. The disk of the mouthpiece contains a one-way valve which allows the user to breathe air in through the disk, but prevents air from flowing out through the disk when the user exhales.

U.S. Pat. No. 4,231,364 to Speshyock describes a respiratory control mouthpiece for insertion in a person's mouth and having a centrally located filtering cartridge and a valve to control the flow of air past the filtering material on inhale and out a separate exhale corridor on exhale so that the exhaled bacterial, virus or otherwise contaminated air is never mixed with the filtered and enhanced air and the filter is kept clean of contaminated air at all times.

Contemporary training products for breathing include the Ultrabreathe ASI7492 Compact Breathing Exerciser and the Power Lung Active Sport Breathing Trainer, both by PowerLung, Inc. of Houston, Tex. Each device is intended to use the principles of resistance training to build breathing muscles and to increase breathing capacity. Each device includes a soft mouthpiece held between the teeth and gums with a bite that creates an airtight seal. Valves associated with inhalation and exhalation can be systematically adjusted to increase resistance as the user's muscles adapt to the loads and stress placed on them. Despite training advantages, both include considerable physical apparatus positioned outside the user's mouth and immediately in front of the user's face, and they are therefore entirely ill-suited for wearing while engaged in any intense physical activity, and positively contraindicated for use in boxing, as they would present a dangerous structure that would be smashed into the user's face with a direct blow.

Accordingly, there remains a need for a mouth guard that can be used for training circular breathing and that can also be employed in full- or limited contact sports, such as ice hockey, field hockey, football, rugby, water polo, European team handball, lacrosse, basketball, wrestling, boxing, mixed martial arts, and the like.

The foregoing background discussion describes the current state of the art of which the present inventor is aware. Reference to, and discussion of, these publications is intended to aid in discharging Applicant's acknowledged duty of candor in disclosing information that may be relevant to the examination of claims to the present invention. However, it is respectfully submitted that none of the above-indicated publications disclose, teach, suggest, show, or otherwise render obvious, either singly or when considered in combination, the invention described and claimed herein. Thus, until the present invention, there has remained needed is an athletic mouth guard suited for use in boxing, wrestling, and martial arts both for training proper breathing and for forcing proper breathing during competition.

### SUMMARY OF THE INVENTION

To provide the protection needed for full contact sports, while also facilitating circular breathing, in its most essential aspect the present invention is a mouth guard and breathing trainer that includes a buccal wall, a lingual wall, a biting surface disposed between the buccal and lingual walls, and a one-way valve centered in the front portion of said mouth guard, wherein when worn by a user, the one-way valve can be urged into an open position through mouth exhalation but cannot be open through mouth inhalation, thereby promoting nose inhalation and mouth exhalation.

In a preferred embodiment, the valve box is formed in the front portion of the mouth guard and extends through an arcuate buccal wall, an arcuate lingual wall spaced apart from the arcuate buccal wall, and a tooth bed disposed between the arcuate lingual wall and arcuate buccal wall. The valve box includes an air passage with an opening in the front portion of the arcuate buccal wall, and the one-way valve is disposed in the valve box opening. The operative element in the valve box includes at least one resilient flap in the form of a petal reed valve, which bends outwardly on exhalation to provide an opening through the air passage, and bends back after exhalation to close the air passage.

In another embodiment, the valve includes two flaps in the form of a two-petal reed valve.

In another embodiment, the valve box comprises an insert that may be selectively placed in a valve insert opening formed in the mouth guard at the time of manufacture. The valve box may then be welded or otherwise permanently secured in the valve insert opening or may be releasably secured in the valve insert opening, thus providing means for cleaning, repairing, and replacing the valve assembly.

In still another embodiment, the valve box is integral with or permanently affixed to the mouth guard and includes a front flap flush with the outer surface of the buccal wall.

In all of the preferred embodiments, all of the structural and operative elements are contained within the mouth of the wearer when in use, and the apparatus is therefore suitable for use in contact sports in which the user might suffer a blow to the head or mouth.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 is an upper left perspective view of a first preferred embodiment of the athletic mouth guard and breathing trainer of the present invention;

FIG. 2 is a cross sectional top plan view thereof, taken along section line 2-2 of FIG. 1;

FIG. 3A is a cross-section side view in elevation thereof, taken along section line 3A-3A of FIG. 1, the view showing the disposition of the operative vale during inhalation;

FIG. 3B is a cross-sectional side view in elevation, showing the disposition of the operative valve during exhalation;

FIG. 4 is an exploded upper left perspective view showing a second preferred embodiment of the present invention, showing the selectively insertable valve box removed from the mouth guard;

FIG. 5A is a cross-sectional top plan view thereof, showing the disposition of the operative valve during inhalation;

FIG. 5B is a cross-sectional top plan view thereof, showing the disposition of the operative valve during exhalation;

FIG. 6A is an upper left perspective view showing a third preferred embodiment of the present invention, showing the disposition of the flap valve during inhalation;

FIG. 6B is an upper left perspective view showing the flap valve during exhalation;

FIG. 6C is a cross-sectional top plan view thereof, showing the operative flap of this species in the close position during inhalation;

FIG. 6D is a cross-sectional side view in elevation showing the operative flap in the open position during exhalation; and

FIG. 6E is a cross-sectional side view in elevation showing the operative flap in the closed position.

### DETAILED DESCRIPTION OF THE INVENTION

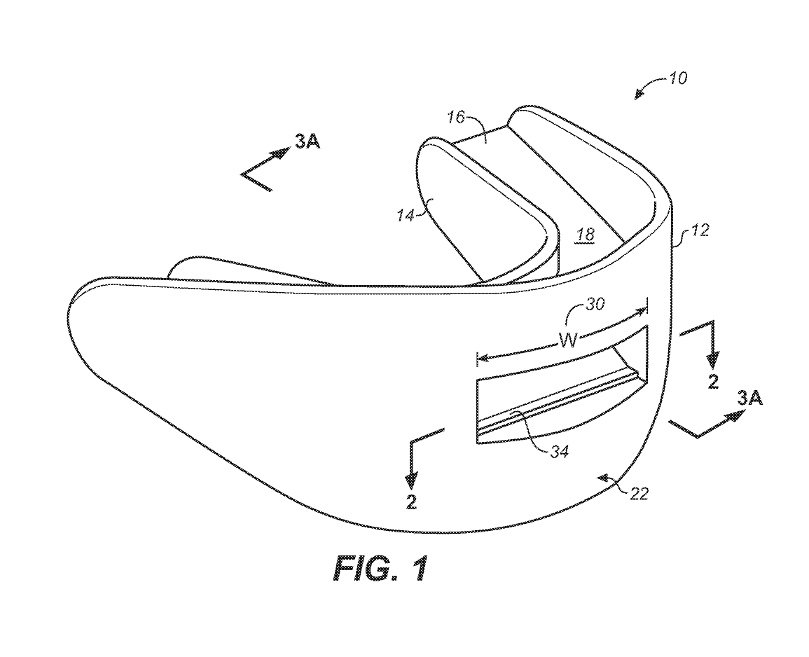

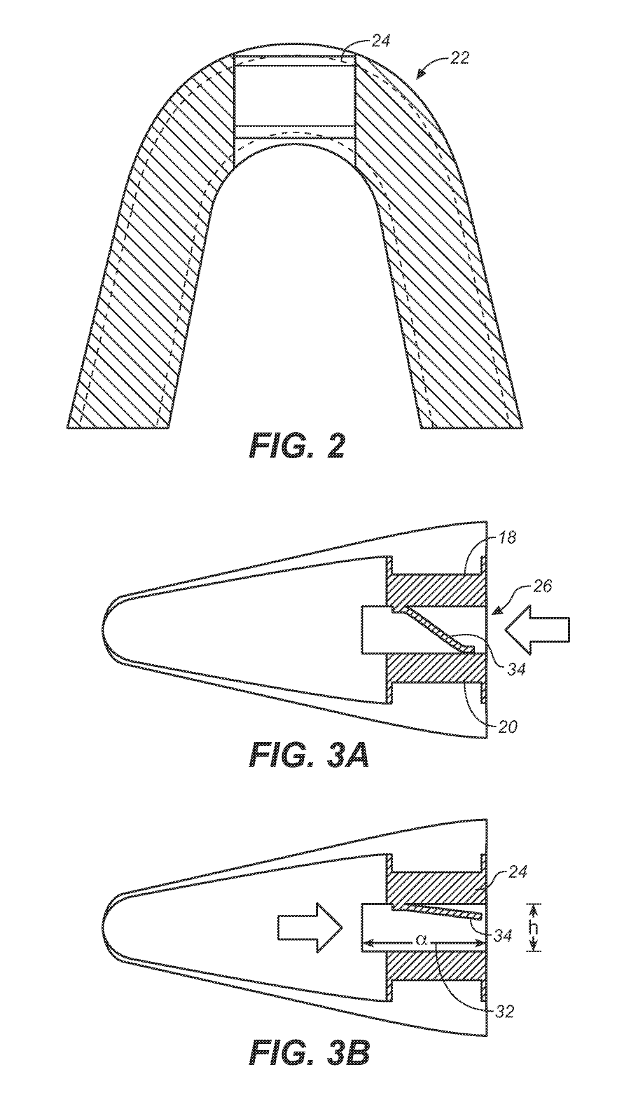

Referring first to FIGS. 1 through 3B, wherein like reference numerals refer to like components in the various views, there is illustrated therein the first preferred embodiment of the new and improved athletic mouth guard and breathing trainer of the present invention, generally denominated 10 herein. These views collectively show that the inventive apparatus includes an arcuate buccal wall 12, an arcuate lingual wall 14 spaced apart from the arcuate buccal wall, and a tooth bed 16 disposed therebetween. The tooth bed provides an upper biting surface 18 and a lower biting surface 20 for the wearer's upper and lower rows of teeth, respectively, and functions as the panel onto which the wearer bites to secure the mouth guard in place.

The mouth guard includes a front portion 22 is proximate the wearer's front teeth when worn. The arcuate buccal wall in this region is generally centered immediately behind the wearer's philtrum and the fubercle of the upper lip and the groove of the lower lip. The front portion includes a valve box 24, that comprises an air passage 26, preferably being a generally box-shaped opening having a height dimension 28 that is smaller than both the width 30 and depth 32 dimensions. The valve box may be molded into the mouth guard or fashioned as an insert for insertion after manufacture of the mouth guard into a box-shaped space adapted for receiving and capturing the valve insert. If manufactured in this manner, the inserted valve box may be welded or glued in the mouth guard to prevent inadvertent loss during use.

The valve box includes a resilient flap 34 forming a single petal reed valve that remains in a down and closed position when not in use or during inhalation (see FIG. 3A). Thus, unless the user loosens his bite and opens his mouth to create a passage for mouth breathing, the mouth guard effectively prevents inhalation through the mouth. As shown in FIG. 3B, the flap can be opened with a sufficiently strong exhalation through the valve insert opening. After the exhalation, the flap returns to the closed position so as to make the user inhale through his nose.

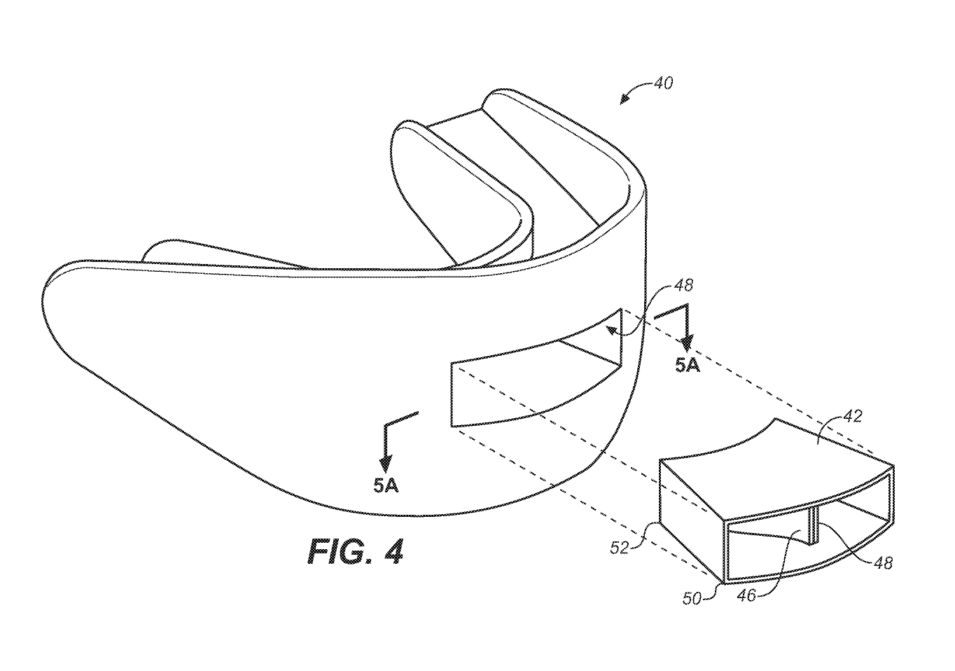

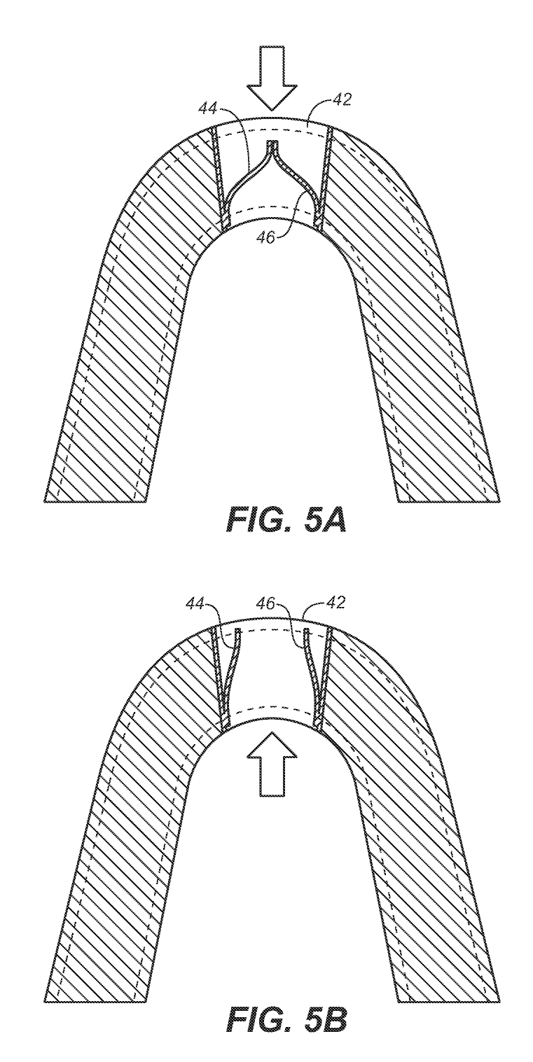

FIGS. 4-5B show a second preferred embodiment 40 of the present invention. The structural elements are identical to those of the first preferred embodiment, except that the valve box is shown here as a valve insert 42 (for insertion as described above) and includes an operative valve having two vertically disposed and opposing flaps 44, 46 forming a two-petal reed valve. As with the first preferred embodiment, the valve flaps open only upon exhalation and close after exhalation to prevent inhalation through the mouth guard, and thereby strongly discourage inhalation through the mouth.

Differing from the first preferred embodiment, in this second preferred embodiment, an insert opening 48 is formed in the mouth guard at the time of manufacture for capturing and retaining the valve insert, as well as the valve insert 42 itself, are each configured with a narrowing taper from the valve insert front 50 to the valve insert back 52, such that the valve insert will not slide rearwardly and further into the wearer's mouth under the force of a blow. This eliminates the choking risk from swallowing this portion of the mouth guard. However, in this embodiment, the valve insert can be removably inserted so as to facilitate replacement of damaged valves.

In models in which the mouth guard is closely conformed to the user's bite characteristics, and thus provided with a custom fit, by first heating the mouthguard in hot water and then biting it in the mouth (the well-known “boil and bite” technique), the valve insert may be removed for the fitting process so as not to suffer any kind of deformation when the mouth guard is heated.

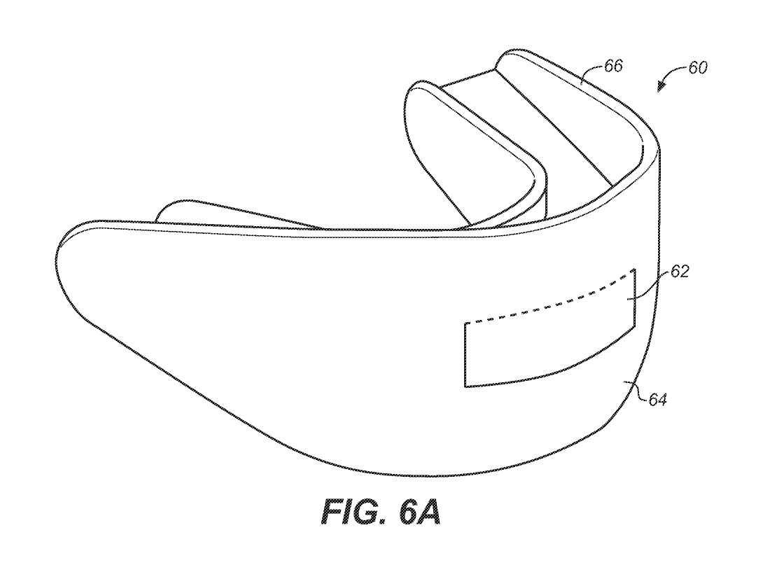

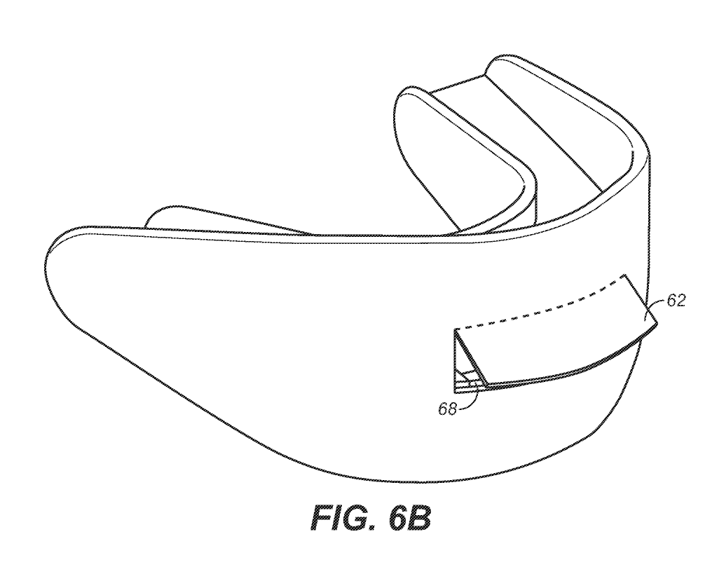

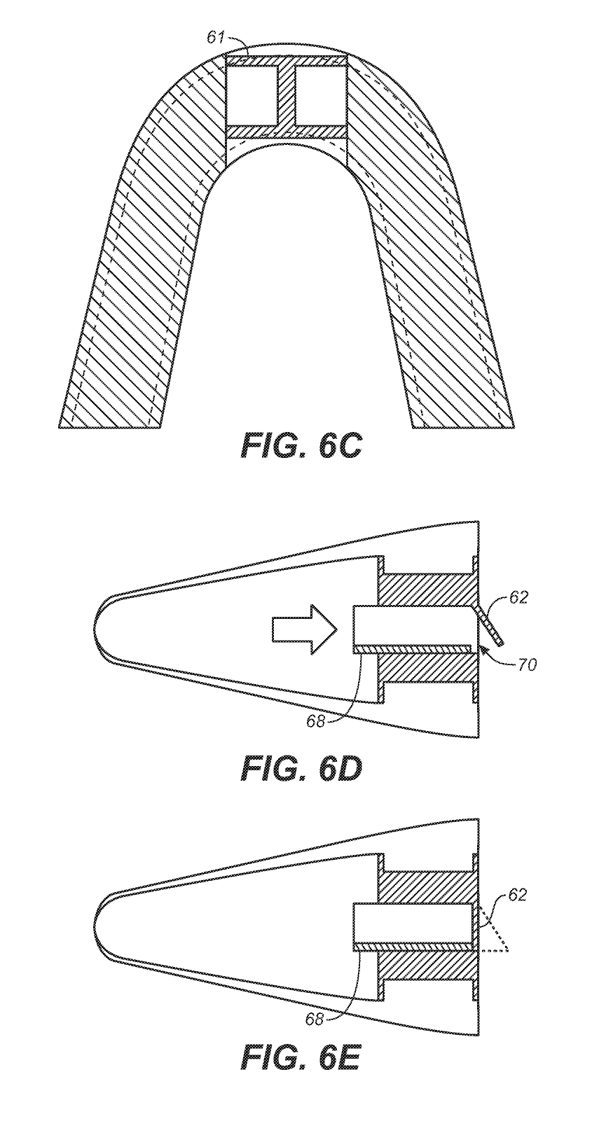

FIGS. 6A-6E show a third preferred embodiment 60 of the present invention. In this embodiment, the valve assembly 61 is either molded or welded into the mouth guard and includes a flap, preferably an upper flap 62, which may be integral with the material forming the front portion 64 of the arcuate buccal wall 66. The flap is flush with the front surface of the buccal wall, and therefore does not present an edge that might irritate the user's oral mucosa. The flap operates under the same principles as the flaps in the valves of the first two preferred embodiments. Depending on the resilience and rigidity of the flap material, the valve may include a stop bed 68 to prevent the flap from folding too far rearward. Furthermore, because the flap is integral with the body of the mouth guard, it remains in place during boil-and-bite fitting. Therefore, to maintain its flush setting, after fitting it may be trimmed to conform to the valve box opening 70.

From the foregoing, it will be clear that each of the embodiments of the inventive mouth guard provide a means to instruct an athlete circular breathing for use when playing a sport. The method entails the steps of providing a mouth guard as described above and instructing the athlete to use the mouth guard in such a manner that he or she does not attempt to bypass use of the one-way valve by breathing around the mouth guard. It should be noted that the mouth guard of the present invention does not include structural barriers that completely prevent breathing through the mouth. Accordingly, if mouth breathing were absolutely necessary, for instance if a nose bleed or injury prevented effective nose breathing, then the mouth guard could be bypassed, if only temporarily. However, proper use of the mouth guard promotes circular breathing when such breathing is possible, as it is highly preferable in even the most vigorous sports.

The above disclosure is sufficient to enable one of ordinary skill in the art to practice the invention. The description also provides the best mode of practicing the invention presently contemplated by the inventor. However, while there is provided herein a full and complete disclosure of the preferred embodiments of this invention, the written description and the drawings do not limit the invention to the exact construction, dimensional relationships, and operation shown and described. Various modifications, alternative constructions, changes and equivalents will readily occur to those skilled in the art and may be employed, as suitable, without departing from the true spirit and scope of the invention.

Therefore, the above description and illustrations should not be construed as limiting the scope of the invention, which is defined instead by the appended claims.

What is claimed as invention is:

1. An athletic mouth guard and breathing trainer, comprising:
an arcuate buccal wall;
an arcuate lingual wall spaced apart from said arcuate buccal wall;
a tooth bed disposed between said arcuate lingual wall and said arcuate buccal wall to provide upper and lower biting surfaces for a wearer's upper and lower rows of teeth;
a valve box including an air passage with an opening in the front portion of said arcuate buccal wall, and a one-way valve disposed in said opening having an open position and a closed position, such that said valve can be opened with a sufficiently strong exhalation through the valve insert opening, and after the exhalation, said valve returns to the closed position that cannot be opened with an inhalation through the mouth, thereby encouraging nose inhalation and circular breathing.

2. The athletic mouth guard and breathing trainer of claim 1, wherein said valve opening has a height dimension smaller than its width and depth dimensions.

3. The athletic mouth guard and breathing trainer of claim 1, wherein said valve box is molded into the mouth guard at the time of manufacture so as to be integral with said arcuate buccal wall, arcuate lingual wall, and said tooth bed.

4. The athletic mouth guard and breathing trainer of claim 1, wherein said valve box is a valve insert and said mouth guard includes a valve insert opening for capturing and retaining said valve insert.

5. The athletic mouth guard and breathing trainer of claim 4, wherein said valve insert opening and said valve insert are each configured with a narrowing taper from the valve insert front to the valve insert back, such that the valve insert will not slide rearwardly and further into the wearer's mouth under the force of a blow.

6. The athletic mouth guard and breathing trainer of claim 4, wherein said valve insert can either be removably inserted into the valve box opening or welded or glued in the mouth guard to prevent inadvertent loss during use.

7. The athletic mouth guard and breathing trainer of claim 1, wherein said one-way valve includes at least one resilient flap.

8. The athletic mouth guard and breathing trainer of claim 6, wherein said at least one resilient flap is a single petal reed valve.

9. The athletic mouth guard and breathing trainer of claim 1, wherein said at least one resilient flap includes two vertically disposed and opposing flaps forming a two-petal reed valve.

10. The athletic mouth guard and breathing trainer of claim 1, wherein said resilient flap is integral with the material forming said front portion of said arcuate buccal wall.

11. The athletic mouth guard and breathing trainer of claim 10, wherein said flap is generally flush with the front surface of said arcuate buccal wall.

12. The athletic mouth guard and breathing trainer of claim 10, wherein said flap includes a stop bed to prevent the flap from folding too far rearward.

13. A mouth guard and breathing training apparatus, comprising:
a buccal wall;
a lingual wall spaced apart from said buccal wall;
a tooth bed disposed between said buccal and lingual walls; and
a one-way valve centered in the front portion of said mouth guard, wherein when worn by a user, said one-way valve can be urged into an open position through mouth exhalation but cannot be open through mouth inhalation, thereby promoting nose inhalation and mouth exhalation.

14. The apparatus of claim 13, wherein the mouth guard is entirely contained within the mouth of the user when in use.

15. The apparatus of claim 13, wherein said one-way valve is disposed in a valve box inserted into a valve insert opening formed in the mouth guard.

16. The apparatus of claim 15, wherein said one-way valve is a reed valve.

17. The apparatus of claim 13, wherein said one-way valve comprises a flap disposed over an air passage opening, said flap being flush with the outer surface of said buccal wall.

18. The apparatus of claim 13, wherein said one-way valve is a reed valve.

19. A method of training circular breathing, comprising the steps of:
(a) providing a mouth guard and breathing training apparatus that includes a buccal wall, a lingual wall spaced apart from said buccal wall, a tooth bed disposed between said buccal and lingual walls, and a one-way valve centered in the front portion of said mouth guard, wherein when worn by a user, said one-way valve can be urged into an open position through mouth exhalation but cannot be open through mouth inhalation, thereby promoting nose inhalation and mouth exhalation;
(b) instructing the athlete to use the mouth guard in such a manner that he or she does not attempt to bypass use of the one-way valve by breathing around the mouth guard.

20. The method of claim 19, wherein the mouth guard provided to the user is entirely contained within the wearer's mouth when in use and is therefore suitable for full contact sports.
